(19) United States
(12) Patent Application Publication
(10) Pub. No.: US 2022/0145398 A1
(43) Pub. Date: May 12, 2022

(54) MIRNA MARKERS FOR COLORECTAL CANCER

(71) Applicant: THE CHINESE UNIVERSITY OF HONG KONG, Shatin, N.T., Hong Kong SAR (CN)

(72) Inventors: Jun YU, Ma On Shan, N.T., Hong Kong SAR (CN); Jao Yiu SUNG, Ma On Shan, N.T., Hong Kong SAR (CN); Qiaoyi LIANG, Tuen Mun, N.T., Hong Kong SAR (CN)

(21) Appl. No.: 17/437,732

(22) PCT Filed: Mar. 10, 2020
(86) PCT No.: PCT/CN2020/078497

§ 371 Date: Sep. 9, 2021

(51) Int. Cl. C12Q 1/6886; C12Q 1/686

(52) U.S. Cl. CPC C12Q 1/6886; C12Q 1/686; C12Q 2600/16; C12Q 2600/158; C12Q 2600/178; C12Q 2600/118

(57) ABSTRACT

The present invention provides novel methods for diagnosing colorectal cancer in a subject or for assessing a colorectal cancer patients likelihood of mortality from the disease by analyzing the quantity of selected miRNA species. Kits, compositions, and devices useful for these methods are also provided.

### RELATED APPLICATIONS

This application claims priority to U.S. Provisional Patent Application No. 62/816,724, filed Mar. 11, 2019, the contents of which are hereby incorporated by reference in the entirety for all purposes.

### BACKGROUND OF THE INVENTION

Colorectal cancer is the third most common cancer worldwide, accounting for about 10% of all cancer cases diagnosed annually. It is a deadly disease with serious impact on human health. During the year of 2012, for instance, 1.4 million new cases of colorectal cancers were diagnosed globally, and nearly 700,000 deaths from the disease were recorded. Incidence of colorectal cancers is substantially higher in developed countries, where more than 65% of cases are found. Men are more likely to suffer from this disease than women.

Diagnosis of colorectal cancer can be challenging. Although family history may provide useful implications for diagnosis, vast majority of the disease (greater than 75-95%) occurs in people with little or no genetic risk. Symptoms of colorectal cancer also can vary significantly, depending on the location of the cancer in the colon, and whether it has spread elsewhere in the body. Depending on how early colorectal cancer is diagnosed, its prognosis can vary from very good to very grim: it is highly curable with surgery when the cancer mass remains confined within the wall of the colon; on the other hand, once colorectal cancer has spread, it is usually not curable, with medical intervention focusing on improving quality of life and alleviating symptoms. On average, the 5-year survival rate in the United States is around 65%.

Because of the high prevalence of colorectal cancer and the vital importance of early diagnosis on patients' life expectancy, there exists an urgent need for new and more effective methods for early diagnosis of colorectal cancer, especially in a non-invasive manner. This invention fulfills this and other related needs.

### BRIEF SUMMARY OF THE INVENTION

The present inventors have discovered that using panels of multiple miRNA markers diagnosis and prognosis of colorectal cancer can be made with improved specificity and sensitivity. More specifically, the use of three, four, or five of miRNA markers miR-92a, miR-21, miR-135b, miR-145, and miR-133a in patient's stool or colon cancer tissue samples allows for reliable determination of disease risk or mortality due to the disease.

As such, in the first aspect, the present invention provides a method for assessing risk for colon cancer in a subject. The method includes the steps of: (a) quantitatively determining expression profile of miR-92a, miR-21, miR-135b, miR-145, and miR-133a in a stool sample taken from the subject; (b) generating a composite score based on the quantity of each of miR-92a, miR-21, miR-135b, miR-145, and miR-133a; and (c) determining whether the subject has an increased risk for colon cancer. In some embodiments, the method further includes a step of performing a fecal immunochemical test (FIT) using the subject's stool sample. A positive FIT test result further indicates increased risk for colon cancer. In some cases, the method is performed on at least two subjects, wherein the first subject having a higher composite score from step (b) is deemed to have a higher risk for colon cancer than the second subject having a lower composite score. In some embodiments, a composite score is generated for a test subject and then compared with a predetermined cut-off value to assess the subject's risk for colon cancer: for example, when the composite score is higher than the cut-off value, the subject is deemed to have an increased risk for colon cancer, which may mean that the subject already has undetected colon cancer or will likely develop the disease in the future. In some cases, when the subject is deemed to have an increased risk for colon cancer, he is further subject to colonoscopy, for example, to confirm whether he has colon cancer, adenoma, or neither. In some cases, step (a) of the method comprises a polynucleotide amplification reaction, such as a polymerase chain reaction (PCR), preferably a reverse transcription PCR (RT-PCR), especially a quantitative RT-PCR.

In a second aspect, the present invention provides a method for assessing a colon cancer patient's likelihood of mortality from colon cancer. The method includes these steps: (a) quantitatively determining expression profile of miR-92a, miR-21, miR-145, and miR-133a in a colon cancer tissue sample taken from the patient; (b) generating a composite score based on the quantity of each of miR-92a, miR-21, miR-145, and miR-133a; and (c) determining whether the patient has an increased risk for mortality from colon cancer. In some embodiments, two or more patients have been tested, and the first patient having a higher composite score is deemed to have a higher risk for mortality from colon cancer than the second patient having a lower composite score. In some embodiments, a composite score is generated for a patient being tested and then compared with a predetermined cut-off value to assess the patient's likelihood of mortality due to colon cancer within a time period (e.g., the next 5 years): for example, when the composite score is less than the cut-off value, the patient is deemed to have at least about 85% chance of survival for the next 5 years or longer; otherwise, the patient has only about 55% chance of survival for the next 5 years. In some embodiments, at the time of testing the patient has already received a diagnosis of colon cancer, for example, at stage I, II, or III. In some embodiments, step (a) of the method comprises a polynucleotide amplification reaction, such as a polymerase chain reaction (PCR), preferably a reverse transcription PCR (RT-PCR), especially a quantitative RT-PCR.

In a third aspect, the present invention provides a method for assessing a colon cancer patient's likelihood of mortality from colon cancer. The method includes these steps: (a) quantitatively determining expression profile of miR-92a, miR-21, and miR-133a in a stool sample taken from the patient; (b) generating a composite score based on the quantity of each of miR-92a, miR-21, and miR-133a; and (c) determining whether the patient has an increased risk for mortality from colon cancer. In some embodiments, two or more patients have been tested, and the first patient having a higher composite score is deemed to have a higher risk for mortality from colon cancer than the second patient having a lower composite score. In some embodiments, a composite score is generated for a patient being tested and then compared with a predetermined cut-off value to assess the patient's likelihood of mortality due to colon cancer within a time period (e.g., the next 5 years): for example, when the composite score is less than the cut-off value, the patient is deemed to have at least about 85% chance of survival for the next 5 years or longer; otherwise, the patient has about 65% chance of survival for the next 5 years. In some embodiments, at the time of testing the patient has already received a diagnosis of colon cancer, for example, at stage I, II, or III. In some embodiments, step (a) of the method comprises a polynucleotide amplification reaction, such as a polymerase chain reaction (PCR), preferably a reverse transcription PCR (RT-PCR), especially a quantitative RT-PCR.

In a fourth aspect, the present invention provides a kit for diagnosis or prognosis of colon cancer in a subject, comprising an agent that specifically and quantitatively detects each one of miR-92a, miR-21, and miR-133a. In some embodiments, the kit comprises an agent that specifically and quantitatively detects each one of miR-92a, miR-21, miR-145, and miR-133a. In some embodiments, the kit comprises an agent that specifically and quantitatively detects each one of miR-92a, miR-21, miR-135b, miR-145, and miR-133a. In some embodiments, the kit includes a primer for reverse transcription of at least one possibly more, up to all five, miRNA. In some embodiments, the kit comprises a set of two oligonucleotide primers for specifically amplifying at least a segment or full length of a reverse-transcribed DNA from any one of the miRNA in an amplification reaction. In some embodiments, the kit comprises a set of oligonucleotide primers for specifically amplifying any one of the miRNA in an RT-PCR. In some embodiments, the kit comprises a set of oligonucleotide primers for specifically amplifying each one of the miRNA in an RT-PCR. In some embodiments, the set of oligonucleotide primers is selected from Table 1. In some embodiments, the agent is a polynucleotide probe that specifically binds the miRNA or a reverse-transcribed DNA from the miRNA. In some embodiments, the agent comprises a detectable moiety. Optionally the kit includes an instruction manual for the user for properly using the kit for its intended diagnosis or prognosis purpose.

In a fifth aspect, the present invention provides use of an agent that specifically and quantitatively detects each one of miR-92a, miR-21, and miR-133a for manufacturing a kit for diagnosis or prognosis of colon cancer in a subject. In some embodiments, the agent includes one that specifically and quantitatively detects each one of miR-92a, miR-21, miR-145, and miR-133a for manufacturing a kit for diagnosis or prognosis of colon cancer in a subject. In some embodiments, the agent includes one that specifically and quantitatively detects each one of miR-92a, miR-21, miR-135b, miR-145, and miR-133a for manufacturing a kit for diagnosis or prognosis of colon cancer in a subject. In some embodiments, five agents each specifically and quantitatively detects one of miR-92a, miR-21, miR-135b, miR-145, and miR-133a are used. In some embodiments, the agents include a set of oligonucleotide primers for specifically amplifying each one of the miRNA in an RT-PCR, e.g., those in Table 1.

### BRIEF DESCRIPTION OF THE DRAWINGS

FIG. 1: (A) Identification CRC-associated miRNAs by analyzing miRNA expression profiling data from three studies. (B) miRNAs selected for further targeted quantification. T, tumor; N, normal; AA, advanced adenoma.

FIG. 2: (A) Fecal levels of the five miRNAs, and their combined score (C-index) by logistic regression model, in a testing cohort of stool samples from 60 CRC patients and 60 control subjects. (B) ROC curve comparison of the diagnostic performances of the three individual miRNAs upregulated in CRC and the combined C-index. (C) Quantitative assessment of individual miRNAs was affected by errors accumulated to template input during experiment, while the combined C-index could tolerate up to ±30% template errors.

FIG. 3: (A) Fecal levels of the three miRNAs that are upregulated in CRC tissues (miR-92a, miR-21 and miR-135b) and C-index in the validation cohort. (B) ROC curve comparison showed that C-index performed significantly better than miR-92a, miR-21 and miR-135b in diagnosing CRC. (C) Correlation of C-index with age, gender, lesion location and TNM stage of CRC patients.

FIG. 4: (A) Fecal level of C-index in a cohort of samples with FIT results, and comparison of FIT, C-index and their combination in diagnosing CRC and advanced adenoma (AA). (B) Comparison of FIT, C-index and their combination in diagnosing CRC according to TNM stage subsets.

FIG. 5: (A) Fecal levels of miR-21, miR-92a or miR-133a could not significantly (B) Ps-index generated from fecal miR-21, miR-92a and miR-133a was significantly associated with patient survival. Ps-index was not associated with age, gender or lesion location of CRC patients, but increased with cancer progression. (C) Multivariate analysis showed that Ps-index was an independent risk factor for poor survival of TNM I-III CRC patients.

FIG. 6: (A) Pm-index, generated from the levels of miR-21, miR-92a, miR-145 and miR-133a in primary CRC, was significantly associated with patient survival. (B) Pm-index was significantly higher in distal cancers than in proximal cancers, and significantly increased with cancer progression. (C) Multivariate analysis showed that Pm-index was an independent risk factor for poor survival of TNM CRC patients.

FIG. 7: Correlation of Cp values of the tested miRNAs in single-target RT-qPCR and multiplex RT-qPCR.

### DEFINITIONS

In this disclosure the terms “colorectal cancer (CRC)” and “colon cancer” have the same meaning and refer to a cancer of the large intestine (colon), the lower part of human digestive system, although rectal cancer often more specifically refers to a cancer of the last several inches of the colon, the rectum. A “colorectal cancer cell” is a colon epithelial cell possessing characteristics of colon cancer and encompasses a precancerous cell, which is in the early stages of conversion to a cancer cell or which is predisposed for conversion to a cancer cell. Such cells may exhibit one or more phenotypic traits characteristic of the cancerous cells.

In this disclosure the term “or” is generally employed in its sense including “and/or” unless the content clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

As used herein, the term “gene expression” is used to refer to the transcription of a DNA to form an RNA molecule encoding a particular protein or the translation of a protein encoded by a polynucleotide sequence. In other words, both mRNA level and protein level encoded by a gene of interest are encompassed by the term “gene expression level” in this disclosure.

In this application, the terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. For the purposes of this application, amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. For the purposes of this application, amino acid mimetics refers to chemical compounds that have a structure that is different from the general chemical structure of an amino acid, but that functions in a manner similar to a naturally occurring amino acid.

Amino acids may include those having non-naturally occurring D-chirality, as disclosed in WO01/12654, which may improve the stability (e.g., half-life), bioavailability, and other characteristics of a polypeptide comprising one or more of such D-amino acids. In some cases, one or more, and potentially all of the amino acids of a therapeutic polypeptide have D-chirality.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

As used in this application, an “increase” or a “decrease” refers to a detectable positive or negative change in quantity from a comparison control, e.g., an established standard control (such as an average level of a pertinent DNA or RNA or protein found in a sample established as a control). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term “inhibiting” or “inhibition,” as used herein, refers to any detectable negative effect on a target biological process, such as RNA transcription, protein expression, cell proliferation, cellular signal transduction, cell proliferation, tumorigenicity, metastatic potential, and recurrence of a disease/condition. Typically, an inhibition is reflected in a decrease of at least 10%, 20%, 30%, 40%, or 50% in target process (e.g., level of a pertinent DNA, RNA, or protein) upon application of an inhibitor, when compared to a control where the inhibitor is not applied.

A “polynucleotide hybridization method” as used herein refers to a method for detecting the presence and/or quantity of a pre-determined polynucleotide sequence based on its ability to form Watson-Crick base-pairing, under appropriate hybridization conditions, with a polynucleotide probe of a known sequence. Examples of such hybridization methods include Southern blot, Northern blot, and in situ hybridization.

“Primers” as used herein refer to oligonucleotides that can be used in an amplification method, such as a polymerase chain reaction (PCR), to amplify a nucleotide sequence based on the polynucleotide sequence corresponding to a gene of interest, e.g., the DNA or RNA sequence of a pertinent bacterial species. Typically at least one of the PCR primers for amplification of a polynucleotide sequence is sequence-specific for that polynucleotide sequence. The exact length of the primer will depend upon many factors, including temperature, source of the primer, and the method used. For example, for diagnostic and prognostic applications, depending on the complexity of the target sequence, the oligonucleotide primer typically contains at least 10, or 15, or 20, or 25 or more nucleotides, although it may contain fewer nucleotides or more nucleotides. The factors involved in determining the appropriate length of primer are readily known to one of ordinary skill in the art. The primers used in particular embodiments are shown in Table 1 of the disclosure where their specific applications are indicated. In this disclosure the term “primer pair” means a pair of primers that hybridize to opposite strands a target DNA molecule or to regions of the target DNA which flank a nucleotide sequence to be amplified. In this disclosure the term “primer site”, means the area of the target DNA or other nucleic acid to which a primer hybridizes.

A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the peptide or used to detect antibodies specifically reactive with the peptide. Typically a detectable label is attached to a probe or a molecule with defined binding characteristics (e.g., a polypeptide with a known binding specificity or a polynucleotide), so as to allow the presence of the probe (and therefore its binding target) to be readily detectable.

The term “cut-off value,” as used in the context of assessing whether a patient being tested has an increased risk for colorectal cancer or whether a colorectal cancer patient has an increased likelihood of mortality from the disease during a future time frame, refers to a pre-determined value in a “composite score” calculated from the quantity of relevant miRNA species (such as miR-92a, miR-21, miR-135b, miR-133a, and miR-145b) in a specific sample type (such as a stool sample or a colorectal cancer tissue sample) processed by a specific method (such as quantitative RT-PCR) using a pre-determined formulation.

The term “amount” as used in this application refers to the quantity of a polynucleotide of interest or a polypeptide of interest (e.g., one or more of miRNA species such as miR-21, miR-92a, miR-135, miR-133a, and miR-145b) present in a sample. Such quantity may be expressed in the absolute terms, i.e., the total quantity of the polynucleotide or polypeptide in the sample, or in the relative terms, i.e., the concentration of the polynucleotide or polypeptide in the sample.

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “subject” or “subject in need of treatment,” as used herein, includes individuals who seek medical attention due to risk of, or actual suffering from, colon cancer. Subjects also include individuals currently undergoing therapy or about to start therapy that seek information useful for making a choice or manipulation of the therapeutic regimen. Subjects or individuals in need of treatment include those that demonstrate symptoms of colon cancer or are at risk of suffering from colon cancer or its symptoms. For example, a subject in need of treatment includes individuals with a genetic predisposition or family history for colon cancer, those that have suffered relevant symptoms in the past, those that have been exposed to a triggering substance or event, as well as those suffering from chronic or acute symptoms of the condition. A “subject in need of treatment” may be any gender and at any age of life. In some cases, the subject may be a patient who has been diagnosed with advanced colorectal cancer (at least stage II, III, IV or even more advanced, e.g., with established distant metastasis).

As used herein, the term “about” denotes a range of value encompassing +/−10% of a pre-determined value. For instance, “about 10” means 9 to 11.

### DETAILED DESCRIPTION OF THE INVENTION

### I. Introduction

The subject matter disclosed generally relates to methods for diagnosis and prognosis of colorectal cancer and markers for these purposes. The invention discloses panels of miRNA predictive and prognostic biomarkers, including 3, 4, or 5 of miRNA species miR-92a, miR-21, miR-135b, miR-133a, and miR-145b, and subsequently methods useful for assessing a test subject's risk of developing colorectal cancer and for assessing a colorectal cancer patient's likelihood of survival from this disease. The present invention also provides miRNA expression detection methods through real-time polymerase chain reaction (PCR) experiment, including the following steps: the total RNA extraction, design miRNA specific primers; miRNA reverse transcription; miRNA real-time PCR quantification. The present invention uses 5 specific miRNAs, miR-92a, miR-21, miR-135b, miR-133a, and miR-145b, as a panel of biomarkers in stool samples for colorectal cancer diagnosis; uses 4 specific miRNAs, miR-92a, miR-21, miR-133a, and miR-145b, as a panel of biomarkers in cancer tissue samples for prospective colorectal cancer mortality; and uses 3 specific miRNAs, miR-92a, miR-21, and miR-133a, as a panel of biomarkers in stool samples for prospective colorectal cancer mortality.

### II. General Methodology

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

The sequence of interest used in this invention, e.g., the polynucleotide sequence of a microRNA, and synthetic oligonucleotides (e.g., primers) can be verified using, e.g., the chain termination method for double-stranded templates of Wallace et al., Gene 16: 21-26 (1981).

III. Acquisition of Samples and Analysis of miRNA

The present invention relates to measuring the level or amount of a specific miRNA found in a sample taken from a patient being tested, for example, a stool sample or colorectal mucosal sample (especially cancer tissue sample), as a means to determine the risk of colorectal cancer in the patient or the likelihood of mortality from colon cancer (or the prospect of survival) after a diagnosis of colorectal cancer has already been made in the patient. Thus, the first steps of practicing this invention are to obtain a sample such as a stool sample or a colon tissue sample (such as colon cancer tissue sample) from a test subject and extract RNA from the sample.

A. Acquisition and Preparation of Samples

A stool sample or a colorectal tissue sample is obtained from a person to be tested for disease risk or for treatment likelihood of survival from the disease. Collection of stool samples can be performed in clinics or patients' homes, whereas collection of colorectal tissue samples is typically performed by way of surgical resection or biopsy such as by colonoscopy. After being obtained, the samples may be stored according to standard procedures prior to further preparation. The analysis of miRNA found in a patient's stool or colorectal sample (which can be taken from either cancer tissue or normal, non-cancerous tissue, especially epithelial tissue such as colon mucosa) according to the present invention may be performed using established techniques. The methods for preparing tissue samples for nucleic acid extraction are well-known among those of skill in the art and described herein.

B. Extraction and Quantitation of RNA

Methods for extracting RNA (containing miRNA) from a biological sample are well-known and routinely practiced in the art of molecular biology (e.g., described by Sambrook and Russell, Molecular Cloning: A Laboratory Manual 3d ed., 2001). The general methods of RNA preparation can be followed, see, e.g., Sambrook and Russell, supra; various commercially available reagents or kits, such as Trizol reagent (Invitrogen, Carlsbad, Calif.), TRIzol LS reagent (Thermo Fisher Scientific, Wilmington, Del.), miRNeasy Mini Kit (Qiagen, Hilden, Germany), Oligotex Direct mRNA Kits (Qiagen, Valencia, Calif.), RNeasy Mini Kits (Qiagen, Hilden, Germany), and PolyATtract® Series 9600™ (Promega, Madison, Wis.), may also be used to obtain mRNA from a biological sample from a test subject. Combinations of more than one of these methods may also be used. It is essential that all contaminating DNA be eliminated from the RNA preparations. Thus, careful handling of the samples, thorough treatment with DNase, and proper negative controls in the amplification and quantification steps should be used.

Once mRNA is extracted from a sample, the amount of any particular miRNA species, such as miR-92a, miR-21, miR-135b, miR-145, and miR-133a, may be quantified. The preferred method for determining the miRNA level is an amplification-based method, e.g., by polymerase chain reaction (PCR), including reverse transcription-polymerase chain reaction (RT-PCR) for RNA quantitative analysis.

Prior to amplification, miRNA must be first reverse transcribed: a DNA copy (cDNA) of the target RNA must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, Minn., 1993; Egger et al., J. Clin. Microbiol. 33:1442-1447, 1995; and U.S. Pat. No. 5,075,212.

The general methods of PCR are well-known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled through a denaturing region, a primer annealing region, and an extension reaction region automatically. Machines specifically adapted for this purpose are commercially available.

### IV. Diagnostic and Prognostic Methods

In order to practice the methods of this invention, a patient stool or colorectal cancer tissue sample is first analyzed to quantify the relevant miRNA species (e.g., miR-92a, miR-21, miR-135b, miR-145, and miR-133a). The quantity of each of the miRNAs is then input into a predetermined formulation to calculate a composite score for each patient.

More specifically, a C-index is calculated for each individual being tested for risk of colon cancer based on the amount of 5 miRNAs, miR-92a, miR-21, miR-135b, miR-145, and miR-133a, found in the individual's stool sample. The higher one's C-index is, the higher risk one has in already suffering from or later developing colorectal cancer. A cut-off value of C-index=2.7417 can be used to generally determine whether an individual is at heightened risk for the disease: if one's C-index is greater than the cut-off value, then he is deemed as having an increased risk for colon cancer. Optionally, the fecal immunochemical test (FIT), a screening test for detecting trace amount of blood in the stool as an early sign of colon cancer, is used concurrently to enhance the detection performance: a positive FIT test result indicates further increased risk for the disease. Calculation of the C-index is performed as follows:

C-index=POWER(2,(0.145*miR145+0.1262*miR133−0.5276*miR92+0.1558*miR21−0.03543*miR135+6.0898))

For assessing a patient, who has already received a diagnosis of colon cancer, a Pm-index is calculated for each patient being tested for likelihood of mortality from colon cancer based on the amount of 4 miRNAs, miR-92a, miR-21, miR-145, and miR-133a, found in the patient's colon cancer tissue sample. The higher one's Pm-index is, the less likely one would survive colorectal cancer within a time period (e.g., the next 1, 2, 3, 4, or 5 years or longer). The comparison between two individuals' Pm-index values thus allows for the determination of which individual is more likely to survive the disease within the next time period of pre-determined length. A cut-off value of Pm-index=1.92 can be used to generally determine whether an individual is likely to die from the disease: if one's Pm-index is greater than the cut-off value, then he is deemed as unlikely to survive colon cancer within the next defined time period. For example, for a patient who has received a diagnosis of colorectal cancer of stage I-IV, if his Pm-index value is less than 1.92, then he has a 79% or greater possibility to live 5 years or longer from the point of diagnosis; otherwise his likelihood of survival for the next 5 years is only about 47%. In another example, a stage colorectal patient having a Pm-index value less than 1.92, his chance of survival for 5 years or longer is about 85%; otherwise his chance of 5-year survival is only about 56%. In general, for a stage I-III patient, a Pm-index below the cut-off value indicates an at least about 85% chance of survival for the next 5 years; otherwise, the chance of survival for the next 5 years is only about 55%. Calculation of the Pm-index is performed as follows:

Pm-index=0.03603483*miR133−0.07733817*miR92−0.34147392*miR145+0.32122176*miR21+3.9243

Similarly, for assessing a patient, who has already received a diagnosis of colon cancer, especially in later stages such as stage II, III, or IV, a Ps-index is calculated for each patient being tested for likelihood of mortality from colon cancer based on the amount of 3 miRNAs, miR-92a, miR-21, and miR-133a, found in the patient's stool sample. The higher one's Ps-index is, the less likely one would survive colorectal cancer within a time period (e.g., the next 1, 2, 3, 4, or up to 5 years). The comparison between two individuals' Ps-index values thus allows for the determination of which individual is more likely to survive the disease within the next time period of pre-determined length. A cut-off value of Ps-index=1.41 can be used to generally determine whether an individual is likely to die from the disease: if one's Ps-index is greater than the cut-off value, then he is deemed as unlikely to survive colon cancer within the next defined time period. For example, for a patient who has received a diagnosis of colorectal cancer of stage I-IV, if his Ps-index value is less than 1.41, then he has an about 77% possibility to live 5 years or longer from the point of diagnosis; otherwise his likelihood of survival for the next 5 years is about 49%. In another example, a stage I-III colorectal patient having a Ps-index value less than 1.41, his chance of survival for 5 years or longer is greater than about 86%; otherwise his chance for 5-year survival is only about 66%. In general, for a stage I-III patient, a Ps-index below the cut-off value indicates an at least about 85% chance of survival for the next 5 years; otherwise, the chance of survival for the next 5 years is much less, only about 65%. Calculation of the Ps-index is performed as follows:

Ps-index=0.2901*miR133−0.1208*miR92−0.2016*miR21+2

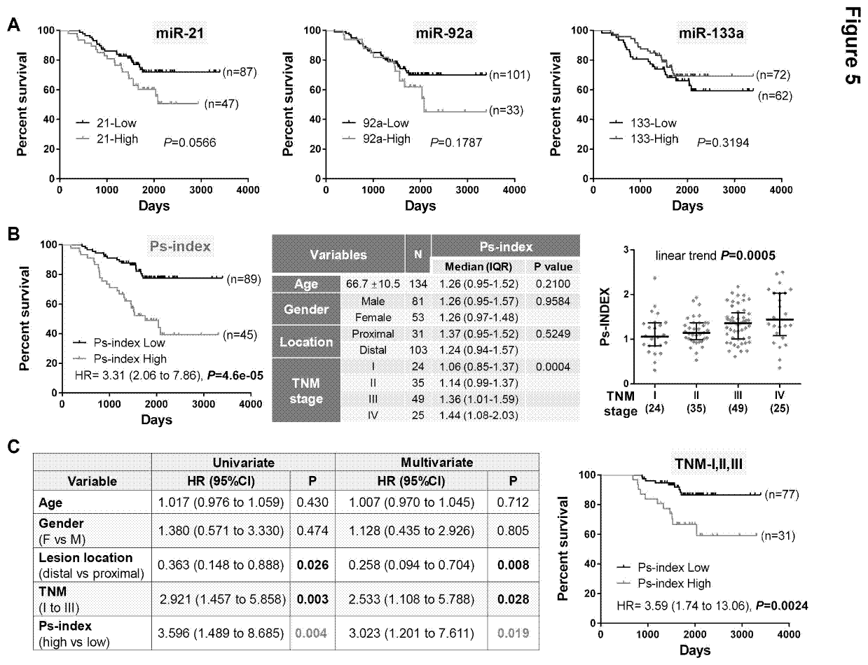

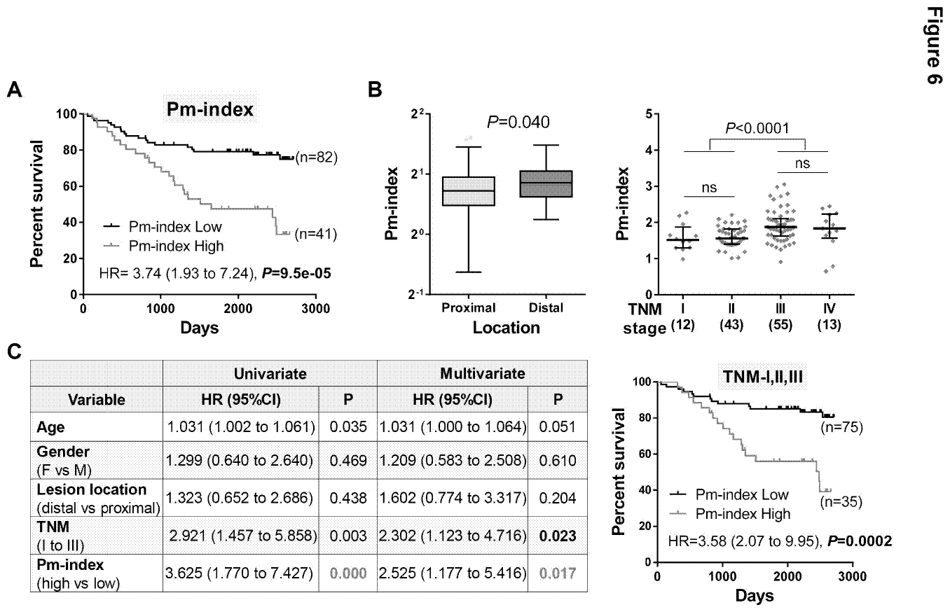

Below is a summary of the experimental data presented in this disclosure, e.g., FIGS. 5 and 6, correlating the Ps and Pm values in individual patients, being higher or lower than the cut-off value, with their 5-year survival:

| | |
|---|---|
| Ps | Pm |

| | Higher | Lower | Higher | Lower |
|---|---|---|---|---|
| I-IV | 49.0% | 77.4% | 47.5% | 79.0% |
| I-III | 66.5% | 86.5% | 56.0% | 85.2% |

### V. Prophylactic Treatment of Colon Cancer

By illustrating the correlation of the presence/amount of specific miRNA species in stool or colorectal mucosal samples and the presence or risk of colon cancer, the present invention provides a preventive measure for prophylactically treating patients who are at an increased risk of later developing colon cancer: upon identification of such at-risk patients, a preventive measure can then be devised for prophylactically treating patients who are at an increased risk of later developing colon cancer.

As used herein, prophylactic treatment of colon cancer encompasses preventing or delaying the onset of one or more of the relevant symptoms of the disease, including reducing mortality or likelihood of disease recurrence among patients who have already received initial treatment.

Upon detecting heightened risk of colorectal cancer in a patient, which is shown by the present inventors by way of analyzing the expression pattern of 5 specific miRNAs in a stool or colon mucosal sample taken from the patient, additional clinical diagnostic methods may be used to confirm or positively establish the presence of colon cancer in the patient. In some cases, the fecal immunochemical test (FIT), a screening test for trace amount of blood in the stool as an early sign of colon cancer, is used concurrently to enhance the detection performance. Further, a more accurate and reliable screening technique (such as colonoscopy) may be used to distinguish whether patient may have adenoma, colon cancer, or neither. As needed, surgical intervention and/or other, non-surgical therapies including chemotherapy, radiotherapy, and immunotherapy may be prescribed by the attending physician. When a patient is deemed to be cancer-free but at risk of later developing colorectal cancer, the patient may be subject to alternative therapies or preventive/monitoring measures, especially among those fitting certain profiles, e.g., those with a family history of cancers especially colon cancer, such that the symptoms of these conditions may be prevented, eliminated, ameliorated, reduced in severity and/or frequency, or delayed in their onset. For example, a physician may prescribe both pharmacological and non-pharmacological treatments such as lifestyle modification (e.g., reduce body weight by 5% or more, assume a healthier life style including following a high fibre/low salt/low fat diet and maintaining a higher level of physical activities such as walking for at least 150 minutes weekly, and undergo regularly scheduled screening/examination such as colonoscopy every 5 years). In some cases, the methods described herein may be practiced on the same patient at a later time, e.g., 5 or 10 years later after the initial testing, to monitor patient status in order to assess any potential change in colorectal cancer risk or presence.

### VI. Kits and Devices

The invention provides compositions and kits for practicing the methods described herein to assess the level of multiple miRNA species (e.g., miR-92a, miR-21, miR-135b, miR-145, and miR-133a) in a sample (e.g., a stool sample or colon mucosal sample) obtained from in a subject. For example, the miRNA levels are measured in a colon mucosal tissue sample taken from a patient, such that the patient may have been treated accordingly, e.g., if the patient is deemed to be at risk of developing colorectal cancer at a later time, the patient will be given the appropriate prophylactic treatment described above and herein.

In some embodiments, kits for carrying out assays for determining the specific miRNA levels typically include reagents useful for carrying out an RT-PCR for the quantitative determination of the miRNAs: at least one oligonucleotide useful for reverse transcription and at least one set of two oligonucleotide primers for PCR to amplify each of the miRNA sequence. In some cases, one or more of the oligonucleotides may be labeled with a detectable moiety. In some cases, a hydrolysis probe is included in the kit to allow instant quantitative measure of amplification product. Typically, the hydrolysis probe has a fluorescent label and a quencher. Table 1 provides some examples of such primers and probes.

Typically, the kits also include information providing an appropriate cut-off value for each of the assay methods. In addition, the kits of this invention may provide instruction manuals to guide users in analyzing test samples and assessing the risk of colorectal cancer or likelihood of mortality from colorectal cancer in a test subject.

In a further aspect, the present invention can also be embodied in a device or a system comprising one or more such devices, which is capable of carrying out all or some of the method steps described herein. For instance, in some cases, the device or system performs the following steps upon receiving a test sample, assessing the risk of colorectal cancer or the likelihood of mortality from colorectal cancer in a patient: (a) determining in the sample the amount or level of each of the specific miRNA species (e.g., miR-92a, miR-21, miR-135b, miR-145, and miR-133a); (b) calculating an index for each sample based on the level of each miRNA species in the sample; (c) comparing the index with a cut-off value or with a second index obtained from a second sample taken from a second patient; and (d) providing an output indicating (1) whether the patient is likely to have colorectal cancer or at risk of later developing the disease and therefore should immediately be given additional diagnostic testing or receive prophylactic treatment or (2) whether the patient, who has received a diagnosis of colorectal cancer, is more likely than the second colorectal cancer patient to survive the cancer within a future time frame (e.g., the next 1, 2, 3, 4, or 5 years, or the next 10, 20, 30, 40, or 50 months). In some cases, the device or system of the invention performs the task of steps (b) through (d), after step (a) has been performed and the amount or concentration of each miRNA from (a) has been entered into the device. Preferably, the device or system is partially or fully automated.

### EXAMPLES

The following examples are provided by way of illustration only and not by way of limitation. Those of skill in the art will readily recognize a variety of non-critical parameters that could be changed or modified to yield essentially the same or similar results.

### Example 1

Background and Aim: MicroRNAs play important roles in the development of colorectal cancer (CRC). Multiple miRNAs have shown to be of diagnostic and/or prognostic value for CRC, but their clinical application is limited due to application of non-targeted methods or single target detection. In this study, the present inventors identified and evaluated the utility of a new panel of miRNAs in the stool-based non-invasive diagnosis and mucosa-based prognosis of CRC.

Experimental Design: Stool samples from 381 subjects (184 CRC, 60 advanced adenoma, and 137 control subjects) and primary CRC tissues from 123 patients were collected. A panel of miRNAs was selected by analyzing genome-wide miRNA expression profiles in CRC. A multiplex RT-qPCR assay and scoring algorithms for diagnosis and prognosis were devised.

Results: By integrative analysis of TCGA small RNA sequencing data and in-house miRNA array data, a panel of 5 miRNAs differentially expressed in CRC tissues compared to normal colon tissues (miR-92a, miR-21, miR-135b, miR-145 and miR-133a) was selected. Then a stem-loop and probe based multiplex RT-qPCR assay was established for the convenient quantification of the five miRNAs. A scoring algorithm to combine all five miRNAs for CRC diagnosis (C-index) was trained by logistic regression on qPCR data from a training cohort of 60 CRC and 60 control fecal samples. Results from the validation cohort showed that, among the individual miRNAs, fecal miR-92a performed best in distinguishing CRC patients from controls, with an area under receiver operating curve (AUROC) of 0.782 (sensitivity=71.7% and specificity=71.5% by Youden's index method). C-index showed significantly improved diagnostic performance compared to individual miRNAs, with an AUROC of 0.849 (P=0.001 vs miR-92a by pairwise comparison of ROCs). At 80.3% specificity, C-index showed a sensitivity of 81.0% for CRC diagnosis, which was further improved by fecal immunochemical test (FIT) to 90.2% (P=0.010). For detection of advanced adenoma (specificity=80.3%), sensitivity of C-index (33.3%) was significantly higher than FIT (16.7%, P=0.035) and was improved to 43.3% by combining with FIT. Moreover, another scoring algorithm for prognosis (Pm-index) was developed to combine mucosal miR-21, miR-92a, miR-145 and miR-133a by proportional-hazards regression models. Kaplan-Meier survival analysis showed that a high Pm-index was significantly associated with shortened survival in CRC patients (HR=3.74 (95% CI: 1.93 to 7.24), P=9.5e-05). Multivariate analysis showed that Pm-index was an independent risk factor for poor survival of CRC patients (HR=2.53 (95% CI: 1.18 to 5.42), P=0.017). Conclusions: This study identified a panel of 5 CRC-related miRNAs and developed a multiplex RT-qPCR and scoring platform that could be conveniently applied in clinical settings for stool-based non-invasive diagnosis and mucosa-based prognosis of CRC.

### INTRODUCTION

Colorectal cancer (CRC) is one of the most common cancers worldwide [1]. The burden of CRC has been increasing tremendously in most of the developed regions in Asia including Hong Kong over the past decades. Early detection of CRC and adenoma has proven to reduce cancer mortality and incidence. The most widely used non-invasive stool test is the fecal immunochemical test (FIT), which shows unsatisfying sensitivities for CRC [2] and is not sensitive for adenoma [3]. In this regard, inclusion of molecular tests targeting sensitive biomarkers to improve the screening performance of FIT is warranted. Colorectal tumorigenesis involves molecular epigenetic and genetic changes in host colorectal epithelial cells. MicroRNAs (miRNAs) have been demonstrated to play important oncogenic or tumor suppressive roles in colorectal tumorigenesis by functional and mechanistic studies, such as miR-92a [4], miR-135b [5], miR-21 [6], miR-145 [7] and miR-133a [8]. As colonic epithelial cells constantly shed into the lumen, molecular changes in these cells can be detected in stool samples. miRNAs have superiorities in serving as stool-based biomarkers for CRC diagnosis. It has been shown that miRNAs remain intact and stable for detection in stool because they are packaged in exosomes [9]. Previous studies showed that miRNA levels in stool samples remain stable for 72 h at room temperature, making it a better marker type than other markers, such as mRNA and methylated DNA, in the stool [10]. Quantification of fecal miRNAs is also of good reproducibility [10]. Multiple miRNAs, such as miR-92a, miR-21 and miR-135b, have been proven individually useful as good fecal diagnostic markers for CRC by several research groups [10-13]. Notably, besides serving as diagnostic markers, miRNAs have also proven useful in prognostic prediction of CRC patients [4, 5, 14-16]. However, as there is no suitable internal control for stool miRNAs [17], relative quantification of targeted miRNAs largely depends on the standard curve method, making the experiment cumbersome. Furthermore, application of single target detection limits the diagnostic or prognostic performance, while quantification of panels of miRNAs by microarray- or sequencing-based methods shows lack of labor efficiency and cost effectiveness. In this study, a panel of five miRNAs were identified and a multiplex RT-qPCR and scoring algorithms were devised for convenient clinical application for non-invasive diagnosis and prognosis of CRC.

### Materials and Methods

### Subjects and Stool Sample Collection

Subjects recruited for fecal sample collection include individuals presenting symptoms such as change of bowel habit, rectal bleeding, abdominal pain or anaemia, and asymptomatic individuals aged 50 or above undergoing screening colonoscopy as in previous metagenomics study [18]. Samples were collected before or one month after colonoscopy. The exclusion criteria included: 1) had any invasive medical intervention within the past 3 months; 2) had a past history of any cancer, or inflammatory disease of the intestine. Subjects were asked to collect stool samples in standardized containers at home, and store the samples in their home −20° C. freezer immediately. Frozen samples were then delivered to the hospitals in insulating polystyrene foam containers and stored at −80° C. immediately until further analysis. Patients were diagnosed by colonoscopic examination and histopathological review of any biopsies taken. Informed consents were obtained from all subjects. Fecal samples were collected from 381 subjects, consisting of 184 patients with CRC (mean age, 66.9±11.2 years; 112 males and 72 females), 60 patients with advanced adenoma (60.0±5.9 years; 40 males and 20 females) and 137 control subjects (58.5±5.8 years; 47 males and 90 females), at the Prince of Wales Hospital, the Chinese University of Hong Kong between 2009 and 2014. The study was conducted with the approval by the Clinical Research Ethics Committee of the Chinese University of Hong Kong.

### Stool RNA Extraction

Stool sample of 200 to 300 mg (wet weight) was added to 1 mL TRIzol LS reagent in a 2-mL tube (Thermo Fisher Scientific, Wilmington, Del.), and homogenized mechanically by RNase-free pestles to deform completely. Chloroform of 200 uL was added to the 2 mL tube and mixed vigorously by shaking for 2 minutes. After incubating at room temperature for 2 minutes, the TRIzol-chloroform mixture was centrifuged for 15 minutes at 12,000 g at 4° C. Upper aqueous phase was then mixed with 1.5 volume of 100% ethanol and purified using the miRNeasy Mini Kit (Qiagen) according to manufacturer's protocol, with a DNase digestion step included by using the RNase-Free Dnase (Qiagen). Total RNA was eluted in 50 uL nuclease-free water. RNA concentration was measured by a NanoDrop One Microvolume UV-Vis Spectrophotometer.

### Tissue Sample Collection and RNA Extraction

Primary colorectal tumors tissues were collected immediately after surgical resection at Peking University Cancer Hospital, Beijing, China (n=123). The specimens were snap-frozen in liquid nitrogen and stored at 80° C. until use. All patients gave informed consent, and the study protocol was approved by the Clinical Research Ethics Committee of the Clinical Research Ethics Committee of Peking University Cancer Hospital. Total RNA was extracted from tissue samples using TRI Reagent according to manufacturer's instruction (Molecular Research Center Inc., Cincinnati, Ohio).

Primer and Probe Design for Reverse Transcription (RT) and qPCR

Primers for RT and primer-probe sets for qPCR were designed by referring to the stem-loop method. Primers and probes specifically targeting the selected miRNAs were listed in Table 1. Each probe carried a 5′ reporter dye FAM (6-carboxy fluorescein), VIC (4,7,2′-trichloro-7′-phenyl-6-carboxyfluorescein), or NED and a 3′ nonfluorescent quencher-minor groove binder (NFQ-MGB). All primers and probes were synthesized at Thermo Fisher Scientific.

TABLE 1
Nucleotide sequences of primers and probes

| Name | Nucleotide sequence (5′->3′) |
|---|---|
| 21-RT | GTCGTATCCAGTGCAGGGTCCGAGGTCTATTCGCACTG |
| | GATACGACTCAACATC (SEQ ID NO: 1) |
| 21-F | GCCGCTAGCTTATCAGACTGATG (SEQ ID NO: 2) |
| 135b-RT | GTCGTATCCAGTGCAGGGTCCGAGGTCTATTCGCACTG |
| | GATACGACTCACATAG (SEQ ID NO: 3) |
| 135b-F | GCCGTATGGCTTTTCATTCCT (SEQ ID NO: 4) |
| 145-RT | GTCGTATCCAGTGCAGGGTCCGAGGTCTATTCGCACTG |
| | GATACGACAGGGATTC (SEQ ID NO: 5) |
| 145-F | GCGTCCAGTTTTCCCAGGA (SEQ ID NO: 6) |
| 92a-RT | GTCGTATCCAGTGCAGGGTCCGAGGTCTATTCGCACT |
| | GGATACGACACAGGCC (SEQ ID NO: 7) |
| 92a-F | GCGTATTGCACTTGTCCCG (SEQ ID NO: 8) |
| 133a-RT | GTCGTATCCAGTGCAGGGTCCGAGGTCTATTCGCACT |
| | GGATACGACCAGCTGG (SEQ ID NO: 9) |
| 133a-F | GCGTTTGGTCCCCTTCAAC (SEQ ID NO: 10) |
| miR-R | GTGCAGGGTCCGAGGTCT (SEQ ID NO: 11) |
| 21-probe | CGCACTGGATACGACTCAACA (SEQ ID NO: 12) |
| 135b-probe | CGCACTGGATACGACTCACAT (SEQ ID NO: 13) |
| 145-probe | CGCACTGGATACGACAGGGAT (SEQ ID NO: 14) |
| 92a-probe | CGCACTGGATACGACACAGG (SEQ ID NO: 15) |
| 133a-probe | CGCACTGGATACGACCAGCTG (SEQ ID NO: 16) |

Reverse Transcription (RT) of miRNAs

A multiplex cDNA synthesis assay was established so that different miRNAs in individual samples could be reverse transcribed together to reduce experimental deviations. RT primer mixture was optimized by comparing RT reactions involving different concentrations of single and multiple primers. TaqMan MicroRNA Reverse Transcription Kit (Life Technology) was used for cDNA synthesis according to manufacturer's protocol, except adjusting reaction system to 15 uL and involving an RT primer mixture (13.3 nM each in final RT reaction) targeting all five selected miRNAs. Total RNA of 100 ng from each sample was applied in each RT reaction, and the 15-uL cDNA product was added with 35 uL nuclease-free water and stored at −20° C. until use.

Multiplex Quantitation miRNAs by qPCR

qPCR reactions of targeted miRNAs were carried out using a combination of primer-probe sets (0.2 μM of each primer and 0.15 μm of each probe), 2 μl cDNA and TaqMan Universal Master Mix II (Life Technology). Thermal cycler parameters, of an ABI QuantStudio™ 7 Flex sequence detection system, were 95° C. 10 min and (95° C. 15 s, 60° C. 1 min)×45 cycles. A positive/reference control and a negative control (H₂O as template) will be included within every experiment. Measurements were performed in triplicates for each sample. qPCR data was analyzed using the Sequence Detection Software (Applied Biosystems) with manual settings of Threshold=0.04, and Baselines from 2-28 cycles for miR-135b and from 2-15 cycles for other miRNAs. Experiments will be disqualified if a Cq value of <42 for negative control is obtained.

### Calculation of the Scores for Diagnosis and Prognosis

Logistic regression model was applied to obtain the C-index for estimating the incidence of CRC as compared to controls. Cox proportional-hazards regression models were applied to fit multiple miRNAs into scores (Pm-index and Ps-index; natural logarithms of hazard ratios) to link with survival outcome. The indexes are calculated as following: C-index=Power [2, +β₁X₁+β₂X₂+β₃X₃+β₄X₄+β₅X₅)], Ps-index=I₂+βₐXₐ+β_{b}X_{b}+β_{c}X_{c}, and Pm=I₃++βᵢXᵢ++βᵢᵢXᵢᵢ+βᵢᵢᵢXᵢᵢᵢ−βᵢᵥXᵢᵥ, where I represented the intercepts, β represented the regression coefficients and X represented the Cp values of the corresponding miRNAs. Compiled MATLAB applications for calculation of the indexes can be downloaded via the links (to be provided).

### Statistical Analyses

The levels of markers (individual miRNAs and other indexes) were all expressed as median and interquartile range [IQR]. The differences in marker levels were determined by Wilcoxon signed-rank test or Mann-Whitney U test. One-way ANOVA multiple comparison with test for linear trend was used to evaluate the changes of marker levels during disease progression (from control to adenoma to cancer). The performance of the markers was analyzed by calculating the area under the receiver-operating characteristic curve (AUROC), and compared using the Delong's test. The best cutoff values were determined by ROC analyses that maximized the Youden index (J=Sensitivity+Specificity−1) [19]). Pairwise comparison of AUROCs for each method/marker was performed using a nonparametric approach [20]. Continuous clinical and pathological variables were compared by T-test, whilst categorical variables were compared by Chi-square test. Spearman's correlation coefficient was used to estimate the association of markers and other factors of interest. Factors independently associated with CRC diagnosis were estimated using univariate and multivariate logistic regression. All tests were done by Graphpad Prism 5.0 (Graphpad Software Inc., San Diego, Calif.), MedCalc Statistical Software version 18.5 (MedCalc Software bvba, Ostend, Belgium; http://www.medcalc.org; 2018) or SPSS statistical package (version 17.0; SPSS, Chicago, Ill.). P<0.05 was taken as statistical significance.

### Results

Identification of 5 CRC-Associated miRNAs by Integrative Analysis of miRNA Expression Profiling Data

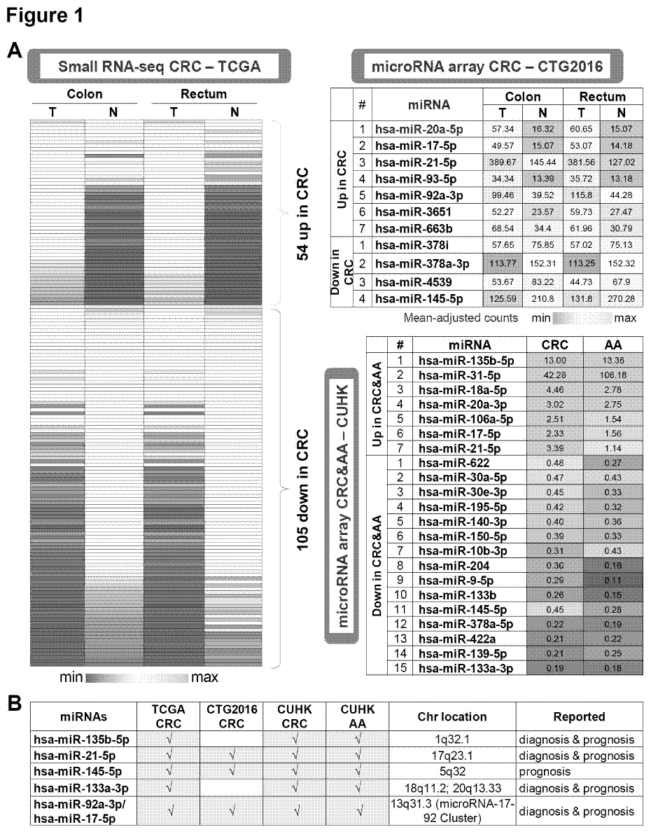

To identify miRNA candidates for CRC diagnosis, miRNA expression profiling data from three studies involving different ethnic groups were analyzed, including small RNA sequencing dataset from The Cancer Genome Atlas (TCGA) study (398 colon cancers with 8 adjacent normal tissues, and 158 rectum cancers with 3 adjacent normal tissues), miRNA array data on colon and rectum cancers conducted by Pellatt et al. (580 colon cancers with 413 adjacent normal tissues, and 341 rectum cancers with 219 adjacent normal tissues) [21], and the miRNA array data on CRC and advanced adenoma conducted by us previously [11] (FIG. 1A). After comparing the expression profiles of tumors with those of controls in each study, miRNAs commonly found to be dysregulated in CRC in the three studies were selected for further testing. As miR-92a and miR-17 are located at the same chromosomal locus, and miR-92a has been proven to be useful for CRC diagnosis by a previous study [10], miR-92a was therefore selected. Finally, five miRNAs were selected for further targeted quantification, including three up-regulated (miR-92a, miR-21 and miR-135b) and two down-regulated (miR-133a and miR-145b) in CRC (FIG. 1B).

Establishment of a Multiplex RT-qPCR Assay for Quantification of the miRNA Panel

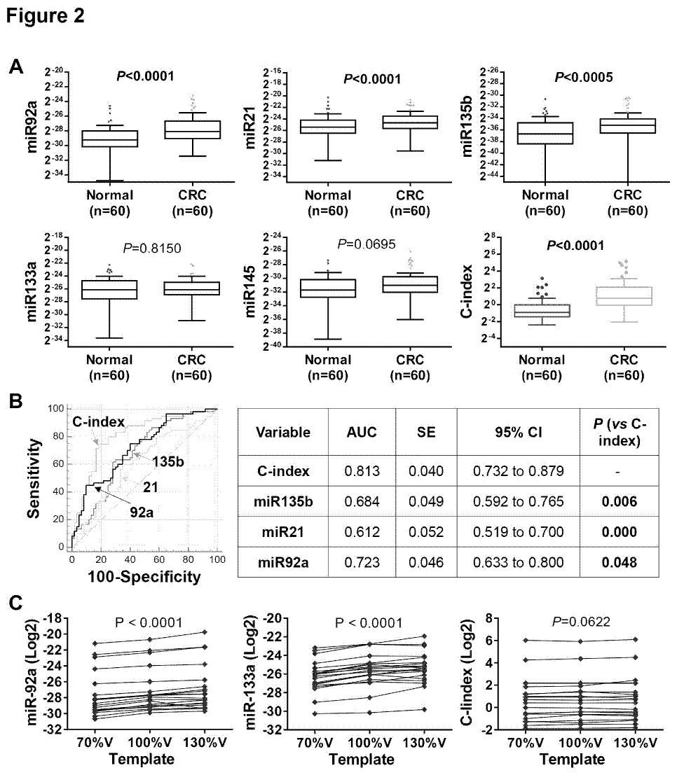

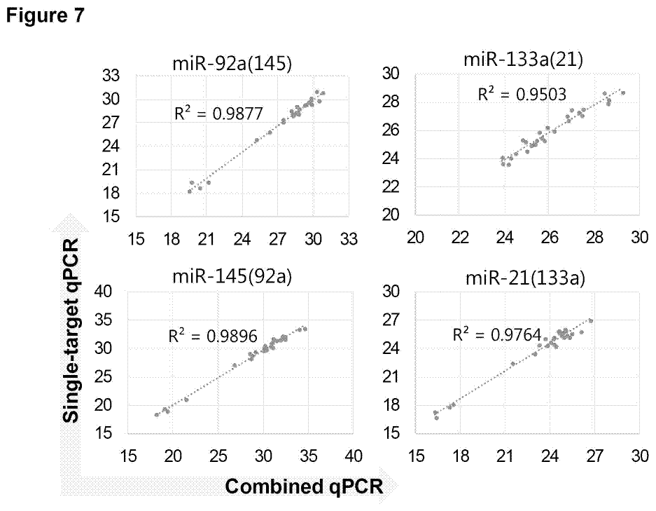

In order to establish a multiplex RT-qPCR assay for convenient and efficient quantitative detection of the 5 miRNAs selected, the stem-loop method [22] was applied for RT primer and qPCR primer-probe design. With the optimized RT-qPCR platform, multiplex reverse transcription to synthesize all 5 cDNAs together could be conducted. Then two duplex qPCR assays (due to lack of commercially available probes for multiplex qPCR) could be conducted, with quantification of target miRNAs by duplex qPCR correlated well with those by singleplex qPCR (FIG. 7). The assays were then tested on 60 CRC and 60 control samples. As expected, the results showed that miR-92a, miR-21 and miR-135b were significantly more abundant in stool samples of CRC patients as compared to control subjects (all P<0.0005), while miR-133a and miR-145 showed no significant difference between cancer patients and control subjects (FIG. 2A).

Combination of the miRNAs Significantly Improved the Diagnostic Performances of Individual miRNAs for CRC

Using the logistic regression model, a C-index based on the 5 miRNAs was generated to discriminate cancer patients from control subjects (FIG. 2A). C-index showed the biggest AUROC as compared to the individual miRNAs in this testing cohort. Pairwise comparison of the ROC curves also showed that the combined C-index was significantly better than the individual miRNAs for CRC diagnosis (all P<0.05; FIG. 2B). The effect of template input on C-index was further evaluated by changing the loading of cDNAs during qPCR to mimic experimental errors, which caused significant proportional changes in the assessment of individual miRNAs. Results showed that, with the inclusion of both up- and down-regulated miRNAs in CRC, the C-index was not significantly affected by template inputs of ±30% deviations (FIG. 2C).

### Performance of the Combined C-Index in the Non-Invasive Diagnosis of CRC

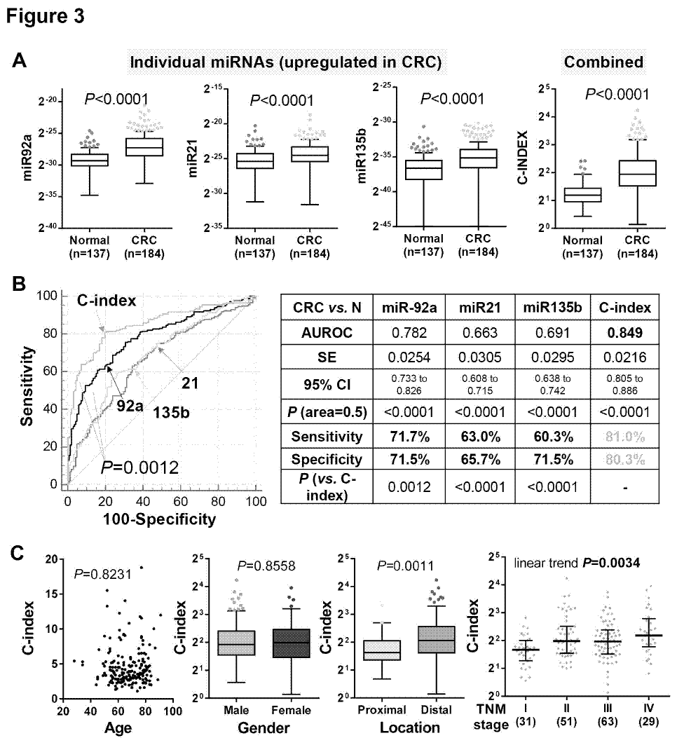

The abundances of the five miRNAs were further examined in stool samples from 184 CRC patients and 137 control subjects. Results confirmed that miR-92a, miR-21 and miR-135b were significantly more abundant in stool samples of CRC patients as compared to control subjects (all P<0.0001; FIG. 3A), while miR-133a and miR-145 showed no difference between CRC patients and control subjects (not shown). Fecal miR-92a showed the best performance in discriminating cancer patients from control subjects among the individual miRNAs, with an AUROC of 0.782. At the best cut-off value, miR-92a showed a sensitivity of 71.7% and specificity of 71.5% (FIG. 3B), which is similar to previous finding [10]. Based on all the 5 miRNAs, the combined C-index showed significantly improved performance for CRC diagnosis (AUROC=0.849) as compared to the individual miRNAs (all P≤0.0012 by pairwise comparison of ROC curves). At the best cut-off value, C-index showed a sensitivity of 81.0% and specificity of 80.3% in diagnosing CRC (FIGS. 3A&B). Correlation analyses showed that C-index was not associated with age or gender of CRC patients, and was significantly in patients with distal cancer as compared to those with proximal cancers. C-index showed a linear trend of increase with cancer progression (FIG. 3C). These results demonstrated that the C-index generated from the miRNA panel may serve as a useful tool for non-invasive diagnosis of CRC.

Combining with FIT Improves the Diagnostic Ability of C-Index for CRC and Advanced Adenoma

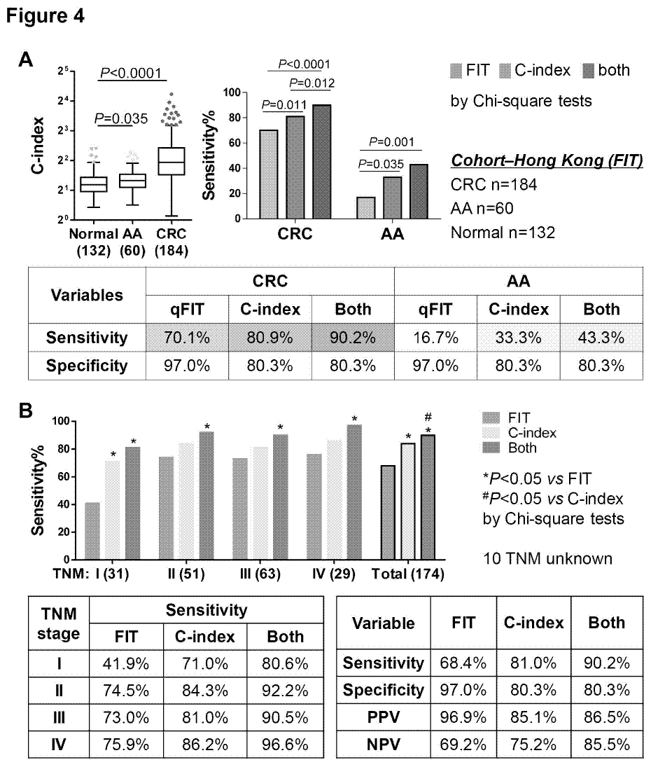

There was a significant increase in C-index in advanced adenoma (AA) patients as compared to control subjects (P=0.035; FIG. 4A), demonstrating that the miRNA panel was also useful in detecting AA. The diagnostic performance of the miRNA panel and FIT were further compared. The C-index was more sensitive than FIT in detecting cancer (80.9% vs 70.1%, P=0.011) and AA (33.3% vs 16.7%, P=0.035). Combination of C-index and FIT further increased the sensitivity to 90.2% for CRC and 43.3% for AA (both P≤0.001 vs FIT) (FIG. 4A). Comparison according to TNM stage subsets was further conducted. The C-index showed a significantly higher sensitivity than FIT for stage I cancers (71.0% vs 41.9%, P=0.021), and their combination showed a further increased sensitivity of 80.6% (P=0.010 vs FIT). Elevated detection rates by C-index compared to FIT were also observed for cancers of other stages, with combination of both showing significantly increased sensitivity than FIT (all P<0.05; FIG. 4B). These results demonstrate that the miRNA panel can be implemented with FIT for the non-invasive diagnosis of CRC and AA patients.

Combination of Fecal miRNAs (Ps-Index) for Prognostic Prediction of CRC Patients

As all the five miRNAs tested have previously been reported for prognosis of CRC [4, 5, 15, 16], it was further evaluated whether fecal abundances of these miRNAs also showed prognostic prediction values. Results showed that none of the individual miRNAs showed significant prediction value for patient survival (all P>0.05; FIG. 5A, miR-135b and miR-145 not shown). However, combination of fecal miR-21, miR-92a and miR-133a by a Cox proportional-hazards regression model, generating a Ps-index (P for prognosis, s for stool), was significantly associated with patient survival. A high Ps-index was significantly associated with poor survival in CRC patients as shown by Kaplan-Meier analysis (n=134; HR=3.31 (2.06-7.86), P<0.0001 by Log-rank test; FIG. 5B). Correlation analyses showed that Ps-index was not associated with age, gender or lesion location, but was significantly associated with TNM staging of CRC patients (P=0.0004, Spearman's rank correlation; FIG. 5B). As stage IV patients with distant metastasis showed much shortened survival than patients of other stages, risk factors associated with survival of stages I to III patients were analyzed. Multivariate Cox regression analysis showed that high Ps-index was an independent risk factor for poor survival of TNM stage I-III CRC patients (HR=3.02 (1.20-7.61), P=0.019; FIG. 5C). Quantification of the miRNA panel in stool samples is of prognostic value for CRC patients.

Combination of Mucosal miRNAs (Pm-Index) for Prognostic Prediction of CRC Patients

As it is feasible to detect biomarkers in primary tumor tissues for prognostic prediction, the five miRNAs in a cohort of 123 primary CRC tissues from Beijing were further examined. Cox proportional-hazards regression analysis showed that combination of miR-92a, miR-21, miR-145 and miR-133a, generating a Pm index (m for mucosa) could serve as a prognostic prediction factor for CRC patient survival. A high Pm-index was significantly associated with poor survival in CRC patients by Kaplan-Meier analysis (HR=3.74 (1.93-7.24), P<0.0001 by Log-rank test; FIG. 6A). Pm-index was not associated with age or gender of CRC patients, but was significantly higher in distal lesions than proximal lesions (P=0.040). Pm-index was significantly higher in TNM stages III and W as compared to TNM stages I and II (P<0.0001; FIG. 6B). Multivariate Cox regression analysis showed that high Pm-index was also an independent risk factor for poor survival of TNM stage I-III CRC patients (HR=2.53 (1.18-5.42), P=0.017; FIG. 6C). These results demonstrate that mucosa-based detection of the miRNA panel may serve as a new tool for prognostication of CRC patients.

### DISCUSSION

In this study, by integrative analysis of small RNA sequencing and miRNA array data from CRC patients of different ethnic groups, a panel of five CRC-related miRNAs, of which the individual miRNAs have been reported to be of diagnostic and prognostic significance, were identified to be of most interest. Then a new quantification platform, involving a series of self-designed primers and probes and an optimized protocol, was developed for multiplex RT-qPCR quantification of all five miRNAs together. Algorithms were then created to calculate scoring indexes for diagnosis and prognosis. A C-index involving fecal abundances of all the 5 miRNAs could distinguish CRC patients from healthy subjects with specificity and sensitivity of >80%, as compared to individual miRNAs of specificity and sensitivity <72%. The Ps-index and Pm-index were devised to combine 3 or 4 of the miRNAs, detected in stool and tissue samples respectively, for prognostication of CRC patients. Both Ps-index and Pm-index were shown to serve as independent risk factors for poor survival of CRC patients. This platform, involving the new miRNA panel and the new quantitative detection and scoring methods, is highly sensitive, specific, and easy to conduct. This platform was proven to be very useful for non-invasive diagnosis of CRC and advanced adenoma, as well as prognosis of CRC patients. This invention can be used directly in clinical implementation.

Aberrant expression of miRNAs has been shown to play important roles during colorectal carcinogenesis. Multiple miRNAs, including the five miRNAs applied in this study, have previously been shown to be useful for diagnosis and prognosis of CRC patients [4, 5, 10, 11, 14-16]. However, as there is no suitable internal control for stool miRNAs, relative quantification of targeted miRNAs largely depends on standard curve method, making the experiment cumbersome. Furthermore, the accuracy of standard curve method relies on the accurate quantification and accurate loading the starting materials, which is practically impossible for stool RNA due to variable contaminants. There is also a lack of consensus on an appropriate reference gene for targeted miRNAs in tissue samples. Efforts have been made to identify suitable endogenous normalization controls for miRNAs, resulting in different controls for different tissue types, such as RNU48, U75 and RNU44 for endometrioid endometrial carcinoma and miR-423 for cervical specimens [23, 24]. Multiple studies have tried to identify proper reference genes for miRNA quantification in CRC, but no consensus on this important but underappreciated issue has been made so far [25-27]. Therefore, current methods employing reference genes or standard curves are unsatisfactory.

As both healthy and diseased colonocytes constantly shed into the lumen, detection of miRNAs upregulated in diseased colonocytes in stool samples may be promising for the early diagnosis of CRC, while miRNAs from healthy colonocytes may be used to ‘normalize’ the relative levels of miRNAs from diseased colonocytes. Similarly, difference between oncogenic miRNAs and tumor suppressive miRNAs detected in the same primary tissue sample may be used as a marker. This new method and indexes were designed based on these rationales. The involvement of both up- and down-regulated miRNAs in CRC and the corresponding scoring algorithms obviate the need for internal controls and/or standard curves that are needed in existing methods.

Findings from previous studies on miRNAs for CRC showed limited direct clinical application values due to the focuses on individual miRNAs by targeted quantification, or based on microarray or sequencing methods that are not cost-efficient in clinical settings. Conventional targeted quantification methods, usually involving a primer-specific RT followed by PCR amplification of an individual miRNA and standard curves or reference genes for data analysis, is time- and material-consuming when multiple miRNAs are considered. Currently commercially available universal cDNA synthesis assay, such as the TaqMan™ Advanced miRNA cDNA Synthesis Kit, helps different miRNAs in individual samples be reverse transcribed at the same time, but it involves cumbersome experimental procedures and pre-amplification may be required before targeted PCR. This protocol of multiplex RT-qPCR significantly improved the cost-efficacy as compared to commercially available methods for quantifying the five target miRNAs.

As the miRNAs were selected based on datasets from different ethnic groups, it is expected that this new platform could be applied in different populations. Validation is needed and different cutoff values may need to be determined before this new platform is clinically implemented in different populations. Besides stools and fresh tissue specimens, the experimental protocol is suitable for total RNA from other sample types such as formalin-fixed paraffin-embedded (FFPE) tissue blocks, serum/plasma, and so on. This platform may also be applied to monitor therapeutic effects/disease recurrence, and further tests on such application values are warranted.

In conclusion, this study established a new miRNA platform involving a new panel of five CRC-associated miRNAs and a well-established multiplex quantification method and scoring algorithms. This platform may serve as a new stool-based tool for non-invasive diagnosis of CRC, to be used alone or together with currently available methods such as FIT. This platform may also serve as new stool-based or tissue specimen-based methods for prognosis of CRC patients.

All patents, patent applications, and other publications, including GenBank Accession Numbers or equivalents, cited in this application are incorporated by reference in the entirety for all purposes.

### REFERENCES

1. Allemani C, Matsuda T, Di Carlo V, Harewood R, Matz M, Niksic M, Bonaventure A, Valkov M, Johnson C J, Esteve J, Ogunbiyi O J, Azevedo E S G, Chen W Q, Eser S, Engholm G, Stiller C A, Monnereau A, Woods R R, Visser O, Lim G H, Aitken J, Weir H K, Coleman M P, Group C W. Global surveillance of trends in cancer survival 2000-14 (CONCORD-3): analysis of individual records for 37 513 025 patients diagnosed with one of 18 cancers from 322 population-based registries in 71 countries. Lancet, 2018. 391(10125): 1023-1075.
2. Lee J K, Liles E G, Bent S, Levin T R, Corley D A. Accuracy of fecal immunochemical tests for colorectal cancer: systematic review and meta-analysis. Ann Intern Med, 2014. 160(3): 171
3. Robertson D J, Lee J K, Boland C R, Dominitz J A, Giardiello F M, Johnson D A, Kaltenbach T, Lieberman D, Levin T R, Rex D K. Recommendations on Fecal Immunochemical Testing to Screen for Colorectal Neoplasia: A Consensus Statement by the U S Multi-Society Task Force on Colorectal Cancer. Gastroenterology, 2017. 152(5): 1217-1237 e3.
4. Zhou T, Zhang G J, Liu Z L, Xia S S, Tian H P. Overexpression of miR-92a correlates with tumor metastasis and poor prognosis in patients with colorectal cancer. International Journal of Colorectal Disease, 2013. 28(1): 19-24.
5. Valeri N, Braconi C, Gasparini P, Murgia C, Lampis A, Paulus-Hock V, Hart J R, Ueno L, Grivennikov S I, Lovat F, Paone A, Cascione L, Sumani K M, Veronese A, Fabbri M, Carasi S, Alder H, Lanza G, Gafa′ R, Moyer M P, Ridgway R A, Cordero J, Nuovo G J, Frankel W L, Rugge M, Fassan M, Groden J, Vogt P K, Karin M, Sansom O J, Croce C M. MicroRNA-135b Promotes Cancer Progression by Acting as a Downstream Effector of Oncogenic Pathways in Colon Cancer. Cancer Cell, 2014. 25(4): 469-483.
6. Zhao J, Zhang Y, Zhao G. Emerging role of microRNA-21 in colorectal cancer. Cancer Biomark, 2015. 15(3): 219-26.
7. Sheng N, Tan G, You W, Chen H, Gong J, Chen D, Zhang H, Wang Z. MiR-145 inhibits human colorectal cancer cell migration and invasion via PAK4-dependent pathway. Cancer Med, 2017. 6(6): 1331-1340.
8. Dong Y, Zhao J, Wu C W, Zhang L, Liu X, Kang W, Leung W W, Zhang N, Chan F K, Sung J J, Ng S S, Yu J. Tumor suppressor functions of miR-133a in colorectal cancer. Mol Cancer Res, 2013. 11(9): 1051-60.
9. Koga Y, Yasunaga M, Moriya Y, Akasu T, Fujita S, Yamamoto S, Matsumura Y. Exosome can prevent RNase from degrading microRNA in feces. J Gastrointest Oncol, 2011. 2(4): 215-22.
10. Wu C W, Ng S S, Dong Y J, Ng S C, Leung W W, Lee C W, Wong Y N, Chan F K, Yu J, Sung J J. Detection of miR-92a and miR-21 in stool samples as potential screening biomarkers for colorectal cancer and polyps. Gut, 2012. 61(5): 739-45.
11. Wu C W, Ng S C, Dong Y, Tian L, Ng S S, Leung W W, Law W T, Yau T O, Chan F K, Sung J J, Yu J. Identification of microRNA-135b in stool as a potential noninvasive biomarker for colorectal cancer and adenoma. Clin Cancer Res, 2014. 20(11): 2994-3002.
12. Rotelli M T, Di Lena M, Cavallini A, Lippolis C, Bonfrate L, Chetta N, Portincasa P, Altomare D F. Fecal microRNA profile in patients with colorectal carcinoma before and after curative surgery. Int J Colorectal Dis, 2015. 30(7): 891-8.
13. Koga Y, Yasunaga M, Takahashi A, Kuroda J, Moriya Y, Akasu T, Fujita S, Yamamoto S, Baba H, Matsumura Y. MicroRNA expression profiling of exfoliated colonocytes isolated from feces for colorectal cancer screening. Cancer Prev Res (Phila), 2010. 3(11): 1435-42.
14. Xia X, Yang B, Zhai X, Liu X, Shen K, Wu Z, Cai J. Prognostic role of microRNA-21 in colorectal cancer: a meta-analysis. PLoS One, 2013. 8(11): e80426.
15. Liu Q, Yang W L, Luo Y S, Hu S C, Zhu L C. Correlation between miR-21 and miR-145 and the incidence and prognosis of colorectal cancer. Journal of Buon, 2018. 23(1): 29-35.
16. Wan T M, Lam C S, Ng L, Chow A K, Wong S K, Li H S, Man J H, Lo O S, Foo D, Cheung A, Yau T, Poon J T, Poon R T, Law W L, Pang R W. The clinicopathological significance of miR-133a in colorectal cancer. Dis Markers, 2014. 2014: 919283.
17. Ren A, Dong Y, Tsoi H, Yu J. Detection of miRNA as non-invasive biomarkers of colorectal cancer. Int J Mol Sci, 2015. 16(2): 2810-23.
18. Yu J, Feng Q, Wong S H, Zhang D, Liang Q, Qin Y, Tang L, Zhao H, Stenvang J, Li Y, Wang X, Xu X, Chen N, Wu W K, Al-Aama J, Nielsen H J, Kiilerich P, Jensen B A, Yau T O, Lan Z, Jia H, Li J, Xiao L, Lam T Y, Ng S C, Cheng A S, Wong V W, Chan F K, Xu X, Yang H, Madsen L, Datz C, Tilg H, Wang J, Brunner N, Kristiansen K, Arumugam M, Sung J J, Wang J. Metagenomic analysis of faecal microbiome as a tool towards targeted non-invasive biomarkers for colorectal cancer. Gut, 2015: Sep. 25. pii: gutjnl-2015-309800.
19. Youden W J. Index for rating diagnostic tests. Cancer, 1950. 3(1): 32-5.
20. DeLong E R, DeLong D M, Clarke-Pearson D L. Comparing the areas under two or more correlated receiver operating characteristic curves: a nonparametric approach. Biometrics, 1988. 44(3): 837-45.
21. Pellatt D F, Stevens J R, Wolff R K, Mullany L E, Herrick J S, Samowitz W, Slattery M L. Expression Profiles of miRNA Subsets Distinguish Human Colorectal Carcinoma and Normal Colonic Mucosa. Clin Transl Gastroenterol, 2016. 7: e152.
22. Chen C, Ridzon D A, Broomer A J, Zhou Z, Lee D H, Nguyen J T, Barbisin M, Xu N L, Mahuvakar V R, Andersen M R, Lao K Q, Livak K J, Guegler K J. Real-time quantification of microRNAs by stem-loop R T-PCR Nucleic Acids Res, 2005. 33(20): e179.
23. Torres A, Torres K, Wdowiak P, Paszkowski T, Maciejewski R. Selection and validation of endogenous controls for microRNA expression studies in endometrioid endometrial cancer tissues. Gynecol Oncol, 2013. 130(3): 588-94.
24. Babion I, Snoek B C, van de Wiel M A, Wilting S M, Steenbergen R D M. A Strategy to Find Suitable Reference Genes for miRNA Quantitative PCR Analysis and Its Application to Cervical Specimens. J Mol Diagn, 2017. 19(5): 625-637.
25. Peltier H J, Latham G J. Normalization of microRNA expression levels in quantitative R T-PCR assays: identification of suitable reference RNA targets in normal and cancerous human solid tissues. RNA, 2008. 14(5): 844-52.
26. Chang K H, Mestdagh P, Vandesompele J, Kerin M J, Miller N. MicroRNA expression profiling to identify and validate reference genes for relative quantification in colorectal cancer. BMC Cancer, 2010. 10: 173.
27. Eriksen A H, Andersen R F, Pallisgaard N, Sorensen F B, Jakobsen A, Hansen T F. MicroRNA Expression Profiling to Identify and Validate Reference Genes for the Relative Quantification of microRNA in Rectal Cancer. PLoS One, 2016. 11(3): e0150593.

What is claimed is:

1. A method for assessing risk for colon cancer in a subject, comprising the steps of:
(a) quantitatively determining expression profile of miR-92a, miR-21, miR-135b, miR-145, and miR-133a in a stool sample taken from the subject;
(b) generating a composite score based on the quantity of each of miR-92a, miR-21, miR-135b, miR-145, and miR-133a; and
(c) determining whether the subject has an increased risk for colon cancer.

2. The method of claim 1, further comprising a step of performing a fecal immunochemical test (FIT).

3. The method of claim 1, when the subject is deemed to have an increased risk for colon cancer, further comprising a step of colonoscopy.

4. The method of claim 1, wherein step (a) comprises a reverse transcription polymerase chain reaction (RT-PCR).

5. The method of claim 4, wherein the polymerase chain reaction (PCR) is a quantitative PCR.

6. The method of claim 4, wherein the PCR is a multiplex PCR amplifying each of reverse transcribed sequence from miR-92a, miR-21, miR-135b, miR-145, and miR-133a.

7. A method for assessing a colon cancer patient's likelihood of mortality from colon cancer, comprising the steps of:
(a) quantitatively determining expression profile of miR-92a, miR-21, miR-145, and miR-133a in a colon cancer tissue sample taken from the patient;
(b) generating a composite score based on the quantity of each of miR-92a, miR-21, miR-145, and miR-133a; and
(c) determining whether the patient has an increased risk for mortality from colon cancer.

8. The method of claim 7, wherein step (a) comprises a reverse transcription polymerase chain reaction (RT-PCR).

9. The method of claim 8, wherein the polymerase chain reaction (PCR) is a quantitative PCR.

10. The method of claim 8, wherein the PCR is a multiplex PCR amplifying each of reverse transcribed sequence from miR-92a, miR-21, miR-145, and miR-133a.

11. A method for assessing a colon cancer patient's likelihood of mortality from colon cancer, comprising the steps of:
(a) quantitatively determining expression profile of miR-92a, miR-21, and miR-133a in a stool sample taken from the patient;
(b) generating a composite score based on the quantity of each of miR-92a, miR-21, and miR-133a; and
(c) determining whether the patient has an increased risk for mortality from colon cancer.

12. The method of claim 11, wherein step (a) comprises a reverse transcription polymerase chain reaction (RT-PCR).

13. The method of claim 12, wherein the polymerase chain reaction (PCR) is a quantitative PCR.

14. The method of claim 13, wherein the PCR is a multiplex PCR amplifying each of reverse transcribed sequence from miR-92a, miR-21, and miR-133a.

15. A kit for diagnosis or prognosis of colon cancer in a subject, comprising an agent that specifically and quantitatively detects each one of miR-92a, miR-21, and miR-133a.

16. The kit of claim 15, comprising an agent that specifically and quantitatively detects each one of miR-92a, miR-21, miR-145, and miR-133a.

17. The kit of claim 15, wherein an agent that specifically and quantitatively detects each one of miR-92a, miR-21, miR-135b, miR-145, and miR-133a.

18. The kit of any one of claims 15-17, comprising a set of oligonucleotide primers for specifically amplifying any one of the miRNA in an RT-PCR.

19. The kit of any one of claims 15-17, comprising a set of oligonucleotide primers for specifically amplifying each one of the miRNA in an RT-PCR.

20. The kit of claim 15, wherein the set of two oligonucleotide primers is selected from Table 1.

21. The kit of claim 15, wherein the agent is a polynucleotide probe that specifically binds the miRNA or a reverse-transcribed DNA from the miRNA.

22. The kit of claim 15, wherein the agent comprises a detectable moiety.

23. The kit of claim 15, further comprising an instruction manual.

24. Use of an agent that specifically and quantitatively detects each one of miR-92a, miR-21, and miR-133a for manufacturing a kit for diagnosis or prognosis of colon cancer in a subject.

25. Use of an agent that specifically and quantitatively detects each one of miR-92a, miR-21, miR-145, and miR-133a for manufacturing a kit for diagnosis or prognosis of colon cancer in a subject.

26. Use of an agent that specifically and quantitatively detects each one of miR-92a, miR-21, miR-135b, miR-145, and miR-133a for manufacturing a kit for diagnosis or prognosis of colon cancer in a subject.

27. The use of any one of claims 24-26, wherein the agent comprises a set of oligonucleotide primers for specifically amplifying each one of the miRNA in an RT-PCR.
